(19) United States
(12) Patent Application Publication
(10) Pub. No.: US 2014/0166015 A1
(43) Pub. Date: Jun. 19, 2014

(54) DUAL MODE MEDICAL OXYGEN DELIVERY SYSTEM

(71) Applicant: Herbert Ross Waggoner, Sebring, FL (US)

(72) Inventor: Herbert Ross Waggoner, Sebring, FL (US)

(21) Appl. No.: 13/717,834

(22) Filed: Dec. 18, 2012

(51) Int. Cl. A61M 16/20; A61M 16/06; A61M 16/00

(52) U.S. Cl. CPC A61M 16/20; A61M 16/0057; A61M 16/0666; A61M 16/06
USPC 128/205.24

(57) ABSTRACT

A dual mode medical oxygen delivery system includes an oxygen supply, a first delivery device, and a second delivery device. A connector includes a valve member and defines a first passage extending from a supply port to a first port and a second passage extending from the supply port to a second port. An oxygen supply conduit interconnects the oxygen supply with the supply port. A first delivery conduit interconnects the first port and the first delivery device, and a second delivery conduit interconnects the second port and the second delivery device. The valve member is movable between a first position corresponding to a first operation mode in which the first passage is fluidly open and the second passage is fluidly blocked, and a second position corresponding to a second operation mode in which the first passage is fluidly blocked and the second passage is fluidly open.

### TECHNICAL FIELD

The present disclosure relates generally to a dual mode medical oxygen delivery system, and more particularly to a medical oxygen delivery system having a connector for maintaining fluid connections between system components and facilitating transitions between first and second operation modes of the system.

### BACKGROUND

Medical oxygen therapy is a treatment that provides patients with additional, or supplemental, oxygen. Normally, the lungs absorb a sufficient amount of oxygen from the air. However, some diseases and conditions, particularly those afflicting the elderly, may prevent the proper intake of oxygen. Thus, medical oxygen therapy may deliver a supplemental amount of oxygen from an oxygen supply, such as a metal cylinder or tank, to a patient. This supplemental amount of oxygen may flow from the oxygen supply, through medical tubing, and may be delivered to the lungs of the patient through an appropriate interface, such as a nasal cannula or face mask. Medical oxygen therapy may be administered in a hospital, nursing home, another medical setting, or at home.

A chronic condition that may also affect patients requiring medical oxygen is sleep apnea. Sleep apnea is a common disorder in which there are one or more pauses in breathing during sleep. These breathing pauses can last from a few seconds to minutes and may occur numerous times each hour. As a result, the quality of sleep may be poor and may result in extreme fatigue and tiredness during the day. Utilizing a continuous positive airway pressure (CPAP) device, or other similar breathing device, while sleeping is the most common treatment for sleep apnea in adults. As is known, a CPAP machine uses a mask that fits over the mouth and/or nose and gently blows air into the throat, with the pressure from the air helping to keep the airway open during sleep. Sleep disorders, including sleep apnea, may be associated with aging in some cases.

As a result, some individuals, including elderly patients, require constant administration of medical oxygen and also require a breathing machine, such as a CPAP machine, during sleep. It is important to note that if the patient has a low resting oxygen level, a chronic pulmonary disease, or other disorder, the delivery of airway pressure using a breathing machine may only serve to treat the sleep apnea and may not raise the overall oxygen level during the night. Thus, inline connectors, or other similar adapters, are provided for connecting an oxygen supply to a breathing machine such that medical oxygen may be supplied to patients in combination with positive airway pressure. For these patients, oxygen is supplied from the oxygen source, through medical tubing, and through a nasal cannula to the patient when the breathing machine is not in use, such as during waking hours. When the breathing machine is to be used, such as during sleep, the patient must remove, or disconnect, the medical tubing connecting the oxygen supply with the nasal cannula and attach, or connect, the medical tubing from the oxygen supply to the inline connector of the breathing machine. This cycle is then repeated on a daily, or even more frequent, basis.

Many of the patients requiring these therapies are elderly and have impaired manual dexterity and strength. Although a technician or family member may assist in initially setting up the system, repeatedly switching between the administration of medical oxygen without the breathing machine and the administration of medical oxygen with the breathing machine may be difficult, if not impossible, for many patients.

The present disclosure is directed toward one or more of the problems or issues set forth above.

### SUMMARY

In one aspect, a dual mode medical oxygen delivery system includes an oxygen supply, a first oxygen delivery device having a first patient interface, and a second oxygen delivery device having a second patient interface. A connector includes a valve member and defines a first fluid supply passage extending from a supply port to a first delivery port and a second fluid supply passage extending from the supply port to a second delivery port. An oxygen supply conduit has a first end fluidly connected to the oxygen supply and a second end fluidly connected to the supply port. A first oxygen delivery conduit has a first end fluidly connected to the first delivery port and a second end fluidly connected to the first oxygen delivery device. A second oxygen delivery conduit has a first end fluidly connected to the second delivery port and a second end fluidly connected to the second oxygen delivery device. The valve member is movable between a first position corresponding to a first operation mode in which the first fluid supply passage is fluidly open and the second fluid supply passage is fluidly blocked, and a second position corresponding to a second operation mode in which the first fluid supply passage is fluidly blocked and the second fluid supply passage is fluidly open.

In another aspect, a method for delivering medical oxygen using a dual mode medical oxygen delivery system includes a step of fluidly connecting a first end of an oxygen supply conduit to an oxygen supply and a second end of the oxygen supply conduit to a supply port of a connector. The method also includes fluidly connecting a first end of a first oxygen delivery conduit to a first delivery port of the connector and a second end of the first oxygen delivery conduit to a first oxygen delivery device having a patient first interface. A first end of a second oxygen delivery conduit is fluidly connected to a second delivery port of the connector and a second end of the oxygen delivery conduit is fluidly connected to a second oxygen delivery device having a second patient interface. The method also includes moving a valve member of the connector to a first position corresponding to a first operation mode in which the first fluid supply passage is fluidly open and the second fluid supply passage is fluidly blocked, and supplying oxygen from the oxygen supply to the first oxygen delivery device while the valve member is in the first position. The method also includes moving the valve member to a second position corresponding to a second operation mode in which the first fluid supply passage is fluidly blocked and the second oxygen delivery conduit is fluidly open, and supplying oxygen from the oxygen supply to the second oxygen delivery device while the valve member is in the second position.

In another aspect, a connector for a dual mode medical oxygen delivery system includes a connector body defining a first fluid supply passage extending from a supply port to a first delivery port and a second fluid supply passage extending from the supply port to a second delivery port. The connector body includes a first connector arm defining a portion of the first fluid supply passage and terminating at the first delivery port, a second connector arm defining a portion of the second fluid supply passage and terminating at the second delivery port, and a third connector arm defining a portion of each of the first fluid supply passage and the second fluid supply passage and terminating at the supply port. A valve member is movable between a first position corresponding to a first operation mode in which the first fluid supply passage is fluidly open and the second fluid supply passage is fluidly blocked, and a second position corresponding to a second operation mode in which the first fluid supply passage is fluidly blocked and the second fluid supply passage is fluidly opened. The first connector arm includes a first set of exterior ribs configured to engage an interior surface of the first end of the first oxygen delivery conduit. The second connector arm includes a second set of exterior ribs configured to engage an interior surface of the first end of the second oxygen delivery conduit. The third connector arm includes a third set of exterior ribs configured to engage an interior surface of the second end of the oxygen supply conduit.

### BRIEF DESCRIPTION OF THE DRAWINGS

FIG. 1 is a schematic view of a dual mode medical oxygen delivery system, according to one embodiment of the present disclosure;

FIG. 2 is a side view of a connector for use with the dual mode medical oxygen delivery system of FIG. 1, with a valve member of the connector shown in a first position corresponding to a first operation mode; and

FIG. 3 is a side view of the connector of FIG. 2, with the valve member shown in a second position corresponding to a second operation mode.

### DETAILED DESCRIPTION

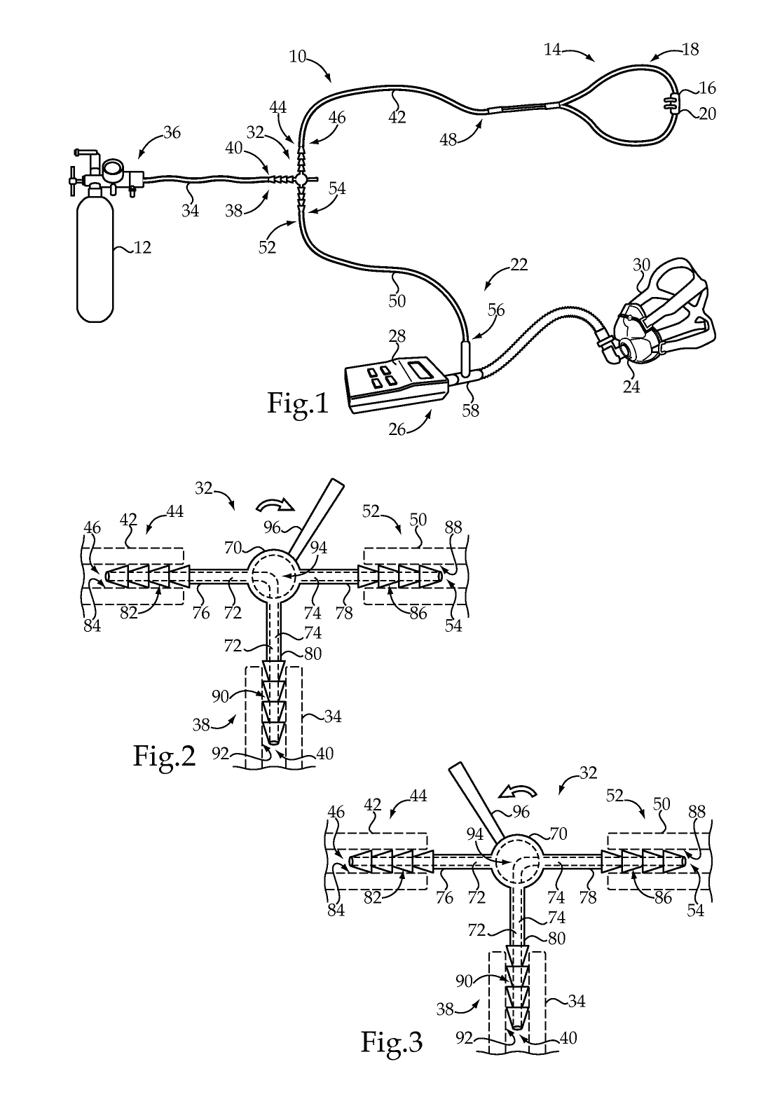

Referring to FIG. 1, a dual mode medical oxygen delivery system 10 may be used in a hospital, nursing home, another medical setting, or a home. The dual mode medical oxygen delivery system 10 includes an oxygen supply 12, or source, for supplying oxygen to a patient. For example, the oxygen supply 12 may include a gas cylinder, tank, or canister varying in size from a small tank, which may be preferred for portable use, to a relatively large tank, which may be designated for non-portable use. Alternatively, the oxygen supply 12 may include an oxygen concentrator or other known source for supplying oxygen to the patient. The oxygen supply 12 may include any necessary valves, regulators, ports, and the like for facilitating the attachment of medical tubing to the oxygen supply 12 for delivery to the patient.

The dual mode medical oxygen delivery system 10 also includes at least two different delivery devices for delivering oxygen from the oxygen supply 12 to the patient. For example, the dual mode medical oxygen delivery system 10 may include a first oxygen delivery device 14 having a first patient interface 16. According to an exemplary embodiment, the first oxygen delivery device 14 may be a supplemental oxygen delivery device 18. In particular, the first oxygen delivery device 14 may be configured for the exclusive administration of oxygen to the patient without the administration of additional, or combined, therapies. Further, the first patient interface 16 may include a nasal cannula 20, which generally consists of two small plastic tubes, or prongs, that are placed in the nostrils of the patient. Alternatively, and as known by those skilled in the art, a mask or other similar interface may be used to deliver supplemental medical oxygen to the patient.

The dual mode medical oxygen delivery system 10 may also include a second oxygen delivery device 22 having a second patient interface 24. The second oxygen delivery device 22 may include the delivery of supplemental medical oxygen to the patient from the oxygen supply 12 in combination with an additional therapy. According to a particular embodiment, the second oxygen delivery device 22 may include a breathing machine 26, for example. Such a breathing machine 26 may include one of a continuous positive airway pressure (CPAP) machine 28, a variable positive airway pressure (VPAP) machine, and a bi-level positive airway pressure (BiPAP) machine, to name a few. The second patient interface 24 may include a mask 30, such as a mask fitting over the nose and mouth, or other similar interface.

It should be appreciated that the first and second oxygen delivery devices 14 and 22, including the respective patient interfaces 16 and 24, are provided as examples only. The dual mode medical oxygen delivery system 10 of the present disclosure may include alternative or additional devices or systems for providing medical oxygen to a patient either alone or in combination with another treatment.

A connector 32 is provided for fluidly connecting components of the dual mode medical oxygen delivery system 10. In particular, the connector 32 may fluidly connect the oxygen supply 12 to both of the first oxygen delivery device 14 and the second oxygen delivery device 22. According to the exemplary embodiment, an oxygen supply conduit 34 has a first end 36 fluidly connected to the oxygen supply 12 and a second end 38 fluidly connected to a supply port 40 of the connector 32. A first oxygen delivery conduit 42 has a first end 44 fluidly connected to a first delivery port 46 of the connector 32 and a second end 48 fluidly connected to the first oxygen delivery device 14. A second oxygen delivery conduit 50 has a first end 52 fluidly connected to a second delivery port 54 of the connector 32 and a second end 56 fluidly connected to the second oxygen delivery device 22 through a connector 58, such as an inline connector. The conduits 34, 42, and 50 may include any known medical tubing suitable for the delivery of medical oxygen and may include single conduits or a plurality of interconnected conduits, or extensions.

Referring now to FIGS. 2 and 3, and according to the exemplary embodiment, the connector 32 includes a connector body 70, which may be a plastic or other suitable material, defining a first fluid supply passage 72 extending from the supply port 40 to the first delivery port 46 and a second fluid supply passage 74 extending from the supply port 40 to the second delivery port 54. As shown, a portion of each of the passages 72 and 74, such as portions adjacent the supply port 40, may be combined. Although a particular embodiment is shown, it should be appreciated that the connector body 70 may divert oxygen from the supply port 40 to a number of different delivery ports, such as first delivery port 46 and second delivery port 54, corresponding to the number of delivery devices, such as first and second oxygen delivery devices 14 and 22, of the dual mode medical oxygen delivery system 10.

According to the exemplary embodiment, the connector body 70 may include a first connector arm 76 defining a portion of the first fluid supply passage 72 and terminating at the first delivery port 46. The connector body 70 may also include a second connector arm 78 defining a portion of the second fluid supply passage 74 and terminating at the second delivery port 54. A third connector arm 80 may define a portion of each of the first fluid supply passage 72 and the second fluid supply passage 74 and may terminate at the supply port 40. The connector arms 76, 78, and 80 may each be configured to facilitate a connection to one of the conduits 34, 42, and 50, as described below.

As shown, the first connector arm 76 may include a first set of exterior ribs 82 configured to engage an interior surface 84 of the first end 44 of the first oxygen delivery conduit 42. The second connector arm 78 may include a second set of exterior ribs 86 configured to engage an interior surface 88 of the first end 52 of the second oxygen delivery conduit 50. The third connector arm 80 may include a third set of exterior ribs 90 configured to engage an interior surface 92 of the second end 38 of the oxygen supply conduit 30. Although ribs 82, 86, and 90 are shown, alternative surface features, including projections, protrusions, and the like may be used to provide an interference fit, or other fit, with the respective conduit 42, 50, or 30. Alternative connection means for facilitating suitable fluid connections and reducing the risk of inadvertent disconnection may also be used without deviating from the scope of the present disclosure. Additional structures for reducing leakage may also be incorporated into the system components of the present disclosure.

A valve member 94 is supported on the connector body 70 and facilitates multiple operation modes of the dual mode medical oxygen delivery system 10. The valve member 94 is rotatable or otherwise movable with respect to the connector body 70, between a first position, as shown in FIG. 2, corresponding to a first operation mode in which the first fluid supply passage 72 is fluidly open and the second fluid supply passage 74 is fluidly blocked, and a second position, as shown in FIG. 3, corresponding to a second operation mode in which the first fluid supply passage 72 is fluidly blocked and the second fluid supply passage 74 is fluidly open. In particular, in the first position, the valve member 94 may be oriented to provide a fluid passage interconnecting the supply port 40 with the first delivery port 46, while also providing a structure blocking fluid passage from the supply port 40 to the second delivery port 54. In the second position, the valve member 94 may be oriented to provide a fluid passage interconnecting the supply port 40 with the second delivery port 54, while also providing a structure blocking fluid passage from the supply port 40 to the first delivery port 46. A lever 96, which may include any of a variety of different shapes and/or configurations, may be supported on the connector body 70 and may be configured to move the valve member 94 between the first position of FIG. 2 and the second position of FIG. 3.

While in the first position (FIG. 2), the valve member 94 fluidly opens the first fluid supply passage 72 to supply oxygen from the oxygen supply 12 to the first oxygen delivery device 14. While oxygen is being supplied from the oxygen supply 12 to the first oxygen delivery device 14, the fluid connection of the second oxygen delivery conduit 50 to the connector 32 and the second oxygen delivery device 22 is maintained (i.e., the components remain interconnected). The lever 96 may be used to switch the valve member 94 from the first position (FIG. 2) corresponding to the first operation mode to the second position (FIG. 3) corresponding to the second operation mode. While in the second position (FIG. 3), the valve member 94 fluidly opens the second fluid supply passage 74 to supply oxygen from the oxygen supply 12 to the second oxygen delivery device 22. While oxygen is being supplied from the oxygen supply 12 to the second oxygen delivery device 22, the fluid connection of the first oxygen delivery conduit 42 to the connector 32 and the first oxygen delivery device 14 is maintained (i.e., the components remain interconnected).

### INDUSTRIAL APPLICABILITY

The present disclosure is generally applicable to medical oxygen delivery systems. Further, the present disclosure is applicable to medical oxygen delivery systems for administering medical oxygen to a patient through a plurality of different medical oxygen delivery devices. Yet further, the present disclosure is directed to a dual mode medical oxygen delivery system permitting the maintenance of established fluid connections of the components of the system by diverting the oxygen supply to a selected one of the plurality of different medical oxygen delivery devices.

An exemplary dual mode medical oxygen delivery system 10 includes an oxygen supply 12 and a first oxygen delivery device 14 having a first patient interface 16. The dual mode medical oxygen delivery system 10 also includes a second oxygen delivery device 22 having a second patient interface 24. A connector 32 is provided for fluidly connecting the oxygen supply 12 to both of the first oxygen delivery device 14 and the second oxygen delivery device 22. The connector 32 includes a connector body 70 defining a first fluid supply passage 72 extending from a supply port 40 to a first delivery port 46 and a second fluid supply passage 74 extending from the supply port 40 to a second delivery port 54.

The dual mode medical oxygen delivery system 10 may be initially set up by a patient, technician, or other individual, in a hospital, nursing home, another medical setting, or a home. To set up the system 10, a first end 36 of an oxygen supply conduit 34 may be fluidly connected to the oxygen supply 12 and a second end 38 of the oxygen supply conduit 34 may be fluidly connected to the supply port 40 of the connector 32. In particular, an interior surface 92 of the second end 38 of the oxygen supply conduit 30 may be secured over a third set of exterior ribs 90 of a third connector arm 80 of the connector body 70. In addition, a first end 44 of a first oxygen delivery conduit 42 may be fluidly connected to the first delivery port 46 of the connector 32 and a second end 48 of the first oxygen delivery conduit 42 may be fluidly connected to the first oxygen delivery device 14. In particular, an interior surface 84 of the first end 44 of the first oxygen delivery conduit 42 may be secured over a first set of exterior ribs 82 of a first connector arm 76 of the connector body 70. Further, a first end 52 of a second oxygen delivery conduit 50 may be fluidly connected to the second delivery port 54 of the connector 32 and a second end 56 of the second oxygen delivery conduit 50 may be fluidly connected to the second oxygen delivery device 22. Specifically, for example, an interior surface 88 of the first end 52 of the second oxygen delivery conduit 50 may be secured over a second set of exterior ribs 86 of a second connector arm 78 of the connector body 70.

To operate the dual mode medical oxygen delivery system 10 in a first operation mode, which may provide for the exclusive administration of medical oxygen, a valve member 94 of the connector 32 may be moved to, or may be maintained at, a first position (FIG. 2), corresponding to the first operation mode, in which the first fluid supply passage 72 is fluidly open and the second fluid supply passage 74 is fluidly blocked. While the valve member 94 is in the first position (FIG. 2), oxygen may be supplied from the oxygen supply 12 to the first oxygen delivery device 14 and ultimately to the patient. The first operation mode may be utilized during waking hours to provide supplemental medical oxygen to the patient, without the administration of additional therapies, such as those described herein.

During sleep, for example, it may desirable to transition the dual mode medical oxygen delivery system 10 from the first operation mode to a second operation mode. To operate the dual mode medical oxygen delivery system 10 in a second operation mode, which may include the combined administration of medical oxygen and an additional therapy, the valve member 94 may be moved to a second position (FIG. 3), corresponding to the second operation mode, in which the first fluid supply passage 72 is fluidly blocked and the second fluid supply passage 74 is fluidly open. While the valve member 94 is in the second position (FIG. 3), oxygen may be supplied from the oxygen supply 12 to the second oxygen delivery device 22. As such, the patient receives medical oxygen in combination with a breathing treatment.

The dual mode medical oxygen delivery system 10 provided herein includes a connector 32 configured for diverting oxygen to a selected one of a plurality of different oxygen delivery devices, such as first and second oxygen delivery devices 14 and 22. As such, the fluid connections of the components of the dual mode medical oxygen delivery system 10 may be maintained, or may remain continuously connected, after the initial install or set up. To repeatedly switch between different operating modes, the valve member 94 of the connector 32 may be repositioned, as described above, to divert the oxygen flow from one medical oxygen delivery device, such as medical oxygen delivery devices 14 and 22, to another. This system 10 may be particularly useful for patients having impaired manual dexterity and strength, who may struggle to repeatedly change system connections to switch between the administration of oxygen through different delivery devices.

It should be understood that the above description is intended for illustrative purposes only, and is not intended to limit the scope of the present disclosure in any way. Thus, those skilled in the art will appreciate that other aspects of the disclosure can be obtained from a study of the drawings, the disclosure and the appended claims.

What is claimed is:

1. A dual mode medical oxygen delivery system, comprising:
an oxygen supply;
a first oxygen delivery device having a first patient interface;
a second oxygen delivery device having a second patient interface;
a connector including a valve member and defining a first fluid supply passage extending from a supply port to a first delivery port and a second fluid supply passage extending from the supply port to a second delivery port;
an oxygen supply conduit having a first end fluidly connected to the oxygen supply and a second end fluidly connected to the supply port;
a first oxygen delivery conduit having a first end fluidly connected to the first delivery port and a second end fluidly connected to the first oxygen delivery device; and
a second oxygen delivery conduit having a first end fluidly connected to the second delivery port and a second end fluidly connected to the second oxygen delivery device;
wherein the valve member is movable between a first position corresponding to a first operation mode in which the first fluid supply passage is fluidly open to supply oxygen from the oxygen supply to the first oxygen delivery device and the second fluid supply passage is fluidly blocked, and a second position corresponding to a second operation mode in which the first fluid supply passage is fluidly blocked and the second fluid supply passage is fluidly open to supply oxygen from the oxygen supply to the second oxygen delivery device.

2. The dual mode medical oxygen delivery system of claim 1, wherein the first oxygen delivery device is a supplemental oxygen delivery device.

3. The dual mode medical oxygen delivery system of claim 2, wherein the first patient interface is a nasal cannula.

4. The dual mode medical oxygen delivery system of claim 2, wherein the second oxygen delivery device includes a breathing machine.

5. The dual mode medical oxygen delivery system of claim 4, wherein the second oxygen delivery device includes one of a continuous positive airway pressure machine, a variable positive airway pressure machine, and a bi-level positive airway pressure machine.

6. The dual mode medical oxygen delivery system of claim 4, wherein the second patient interface is a mask.

7. The dual mode medical oxygen delivery system of claim 1, wherein the connector includes a first connector arm defining a portion of the first fluid supply passage and terminating at the first delivery port, a second connector arm defining a portion of the second fluid supply passage and terminating at the second delivery port, and a third connector arm defining a portion of each of the first fluid supply passage and the second fluid supply passage and terminating at the supply port.

8. The dual mode medical oxygen delivery system of claim 7, wherein each of the first connector arm, the second connector arm, and the third connector arm includes exterior ribs.

9. The dual mode medical oxygen delivery system of claim 1, further including a lever supported on the connector and configured to move the valve member between the first position and the second position.

10. A method for delivering medical oxygen using a dual mode medical oxygen delivery system, the method comprising steps of:
fluidly connecting a first end of an oxygen supply conduit to an oxygen supply and a second end of the oxygen supply conduit to a supply port of a connector;
fluidly connecting a first end of a first oxygen delivery conduit to a first delivery port of the connector and a second end of the first oxygen delivery conduit to a first oxygen delivery device having a first patient interface;
fluidly connecting a first end of a second oxygen delivery conduit to a second delivery port of the connector and a second end of the second oxygen delivery conduit to a second oxygen delivery device having a second patient interface;
moving a valve member of the connector to a first position corresponding to a first operation mode in which the first fluid supply passage is fluidly open and the second fluid supply passage is fluidly blocked;
supplying oxygen from the oxygen supply to the first oxygen delivery device while the valve member is in the first position;
moving the valve member to a second position corresponding to a second operation mode in which the first fluid supply passage is fluidly blocked and the second fluid supply passage is fluidly open; and
supplying oxygen from the oxygen supply to the second oxygen delivery device while the valve member is in the second position.

11. The method of claim 10, further including:
maintaining a fluid connection of the second oxygen delivery conduit to the connector and the second oxygen delivery device while the valve member is in the first position; and
maintaining a fluid connection of the first oxygen delivery conduit to the connector and the first oxygen delivery device while the valve member is in the second position.

12. The method of claim 11, wherein the step of supplying oxygen from the oxygen supply to the first oxygen delivery device includes supplying oxygen to a supplemental oxygen delivery device.

13. The method of claim 12, wherein the step of supplying oxygen from the oxygen supply to the first oxygen delivery device further includes supplying oxygen to a nasal cannula.

14. The method of claim 12, wherein the step of supplying oxygen from the oxygen supply to the second oxygen delivery device includes supplying oxygen to a breathing machine.

15. The method of claim 14, wherein the step of supplying oxygen from the oxygen supply to the second oxygen delivery device further includes supplying oxygen to one of a continuous positive airway pressure machine, a variable positive airway pressure machine, and a bi-level positive airway pressure machine.

16. The method of claim 15, wherein the step of supplying oxygen from the oxygen supply to the second oxygen delivery device further includes supplying oxygen to a mask.

17. The method of claim 10, further including:
fluidly connecting the first end of the first oxygen delivery conduit to a first connector arm of the connector;
fluidly connecting the first end of the second oxygen delivery conduit to a second connector arm of the connector; and
fluidly connecting the second end of the oxygen supply conduit to a third connector arm of the connector.

18. The method of claim 17, further including:
engaging a first set of exterior ribs of the first connector arm with an interior surface of the first oxygen delivery conduit;
engaging a second set of exterior ribs of the second connector arm with an interior surface of the second oxygen delivery conduit; and
engaging a third set of exterior ribs of the third connector arm with an interior surface of the oxygen supply conduit.

19. A connector for a dual mode medical oxygen delivery system, the dual mode medical oxygen delivery system including: an oxygen supply; a first oxygen delivery device having a first patient interface; a second oxygen delivery device having a second patient interface; an oxygen supply conduit having a first end fluidly connected to the oxygen supply and a second end; a first oxygen delivery conduit having a first end and a second end fluidly connected to the first oxygen delivery device; and a second oxygen delivery conduit having a first end and a second end fluidly connected to the second oxygen delivery device, the connector comprising:
a connector body defining a first fluid supply passage extending from a supply port to a first delivery port and a second fluid supply passage extending from the supply port to a second delivery port, wherein the connector body includes a first connector arm defining a portion of the first fluid supply passage and terminating at the first delivery port, a second connector arm defining a portion of the second fluid supply passage and terminating at the second delivery port, and a third connector arm defining a portion of the first fluid supply passage and the second fluid supply passage and terminating at the supply port; and
a valve member movable between a first position corresponding to a first operation mode in which the first fluid supply passage is fluidly open and the second fluid supply passage is fluidly blocked, and a second position corresponding to a second operation mode in which the first fluid supply passage is fluidly blocked and the second fluid supply passage is fluidly open;
wherein the first connector arm includes a first set of exterior ribs configured to engage an interior surface of the first end of the first oxygen delivery conduit;
wherein the second connector arm includes a second set of exterior ribs configured to engage an interior surface of the first end of the second oxygen delivery conduit;
wherein the third connector arm includes a third set of exterior ribs configured to engage an interior surface of the second end of the oxygen supply conduit.

20. The connector of claim 19, further including a lever supported on the connector body and configured to move the valve member between the first position and the second position.
